(19) United States
(12) Patent Application Publication
(10) Pub. No.: US 2022/0061762 A1
(43) Pub. Date: Mar. 3, 2022

(54) HOME-STYLE AND MULTI-FUNCTIONAL TESTING HEARING-AID DEVICE AND METHOD THEREOF

(71) Applicant: AWNT LIMITED, New Taipei (TW)

(72) Inventors: Yi-Chang Liu, New Taipei (TW); Li-Min Sun, Beijing (CN)

(21) Appl. No.: 17/466,120

(22) Filed: Sep. 3, 2021

(30) Foreign Application Priority Data
Sep. 3, 2020 (TW) 109130253

(51) Int. Cl. A61B 5/00; A61B 5/01; A61B 5/11; A61B 5/0205; H04R 25/00; G08B 21/18

(52) U.S. Cl. CPC A61B 5/6815; A61B 5/01; A61B 5/1117; A61B 5/0205; A61B 5/02438; A61B 5/7267; A61B 5/1122; H04R 25/65; G08B 21/182; A61B 5/746

(57) ABSTRACT

A home-style and multi-functional testing hearing-aid device and a method thereof are provided. The falling prevention disposed in the case utilizes a deep learning method to judge the movement status of the case based on a plurality of conventional movement trajectories. The falling prevention generates a falling prevention warning signal when the movement status does not conform to the plurality of conventional movement trajectories. The warning device receives the falling prevention warning signal and executes a warning operation. In addition, the present disclosure also provides other functions such as temperature sensing, pulse measurement and blood pressure measurement at the same time. Accordingly, the present disclosure provides a hearing-aid device with multiple functions, so that the user can use it based on their required function and the convenience to the user can be increased.

### CROSS-REFERENCE TO RELATED APPLICATION

This application claims the benefit of Taiwan Patent Application No. 109130253, filed on Sep. 3, 2020, in the Taiwan Intellectual Property Office, the disclosure of which is incorporated herein in its entirety by reference.

### BACKGROUND OF THE INVENTION

### 1. Field of the Invention

The present invention generally relates to a hearing-aid device, in particular to a home-style and multi-functional testing hearing-aid device and a method thereof, which may provide falling prevention, warning and other functions.

### 2. Description of the Related Art

When the human beings get older, the functions of various organs will gradually degenerate. Thus, in order to improve the problems, such as the inability to hear the external sounds clearly due to the hearing degeneration, a hearing-aid device is currently used to hang on the people's ears. After the processes of removing the noise or amplifying the sound, the external sounds are output to the ear, so that people can clearly hear the external sound.

Indeed, the hearing-aid device may help the hearing impaired to improve their hearing impairment, and thus improve the ability to communicate with others. However, due to the people who need hearing-aid device are usually older, and the conventional hearing-aid device just simply amplifies the received sounds or performs the related process without providing other functions, the conventional hearing-aid device has a defect that the function is single. That is, the conventional hearing-aid device only satisfies the needs of the hearing impaired to communicate with others.

Therefore, there is an urgent need to provide a hearing-aid device that can meet the functions required by the elder people and it is convenient to the users. The problems existing in the prior art can be solved.

### SUMMARY OF THE INVENTION

The objective of the present disclosure provides a home-style and multi-functional testing hearing-aid device, which utilizes a falling prevention device in a case, and a deep learning method is utilized to judge a movement status based on a plurality of conventional movement trajectories. When the falling prevention device judges that the movement status does not conform to the plurality of conventional movement trajectories, the falling prevention device generates a falling prevention warning signal. At this time, a warning device receives the falling prevention warning signal and executes a warning operation. In addition, the present disclosure also provides functions such as temperature sensing, pulse measurement, blood pressure measurement and so on. Accordingly, the present disclosure provides a multi-functional hearing-aid device, which allows the users to use their required functions and to increase the convenience to the users.

To achieve the foregoing objective, the present disclosure provides a home-style and multi-functional testing hearing-aid device, which is hanging on an ear including a case, a sound receiver, a processor, an output device, a falling prevention device and a warning device. The sound receiver is disposed in the case and receives a sound signal from outer range of the case. The processor is disposed in the case and is connected with the sound receiver to receive the sound signal, and the sound signal is amplified or amplified and frequency shifted to generate an output sound signal. The output device is disposed in the case and is connected with the processor to receive and output the output sound signal to the ear. The falling prevention device is disposed in the case and is connected with the processor. The falling prevention device records movements of the case, and a deep learning method is utilized to generate a plurality of conventional movement trajectories based on lines collected by recording the movements of the case and to judge a movement status of the case based on the plurality of conventional movement trajectories. When the movement status does not conform to the plurality of conventional movement trajectories, the falling prevention device generates a falling prevention warning signal and sends the falling prevention warning signal to the processor. When the falling prevention device judges that the movement status conforms to any one of the plurality of conventional movement trajectories, the falling prevention device continues to judge other movement status. The warning device is disposed in the case and is connected with the processor to receive the falling prevention warning signal. The warning device executes a warning operation when the warning device receives the falling prevention warning signal.

Preferably, the home-style and multi-functional testing hearing-aid device further includes a temperature sensor, which is disposed on outer surface of the case and is connected with the processor. The temperature sensor is in contact or adjacent to the ear to obtain temperature information and the temperature sensor judges the temperature information based on a temperature threshold value. When a temperature value of the temperature information exceeds the temperature threshold value, the temperature sensor generates a temperature warning signal and the temperature warning signal is sent to the processor. When the temperature value of the temperature information does not exceed the temperature threshold value, the temperature sensor continues to judge other temperature information. Wherein the warning device executes the warning operation when the warning device receives the temperature warning signal.

Preferably, the home-style and multi-functional testing hearing-aid device further includes a pulse sensor, which is disposed on outer surface of the case and is connected with the processor. The pulse sensor is in contact to the ear to obtain pulse information and the pulse sensor judges the pulse information based on a normal heart rate range. When a pulse value of the pulse information exceeds the normal heart rate range, the pulse sensor generates a pulse warning signal and the pulse warning signal is sent to the processor. When the pulse value of the pulse information does not exceed the normal heart rate range, the pulse sensor continues to judge other pulse information. Wherein the warning device executes the warning operation when the warning device receives the pulse warning signal.

Preferably, the home-style and multi-functional testing hearing-aid device further includes a register, which is disposed in the case and is connected with the processor to store the sound signal, the output sound signal, the plurality of conventional movement trajectories, the movement status, the falling prevention warning signal or a combination of above signals.

Preferably, the home-style and multi-functional testing hearing-aid device further includes a blood pressure sensor, which is disposed on outer surface of the case and is connected with the processor. The blood pressure sensor includes a pressure component and the pressure component presses any portion of the ear to obtain a blood pressure value and the blood pressure sensor judges the blood pressure value based on a normal blood pressure range. When the blood pressure value exceeds the normal blood pressure range, the blood pressure sensor generates a blood pressure warning signal and the blood pressure warning signal is sent to the processor. When the blood pressure value does not exceed the normal blood pressure range, the blood pressure sensor continues to judge other blood pressure value.

Preferably, the home-style and multi-functional testing hearing-aid device further includes a blood pressure sensor, which is disposed on outer surface of the case and is connected with the processor. The blood pressure sensor includes a sound receiving component and the sound receiving component is in contact to any portion of the ear to obtain a blood pressure frequency signal. The blood pressure sensor converts the blood pressure frequency signal to a blood pressure value and the blood pressure sensor judges the blood pressure value based on a normal blood pressure range. When a blood pressure value exceeds the normal blood pressure range, the blood pressure sensor generates a blood pressure warning signal and the blood pressure warning signal is sent to the processor. When the blood pressure value does not exceed the normal blood pressure range, the blood pressure sensor continues to judge other blood pressure value.

Preferably, the warning device executes the warning operation when the warning device receives the blood pressure warning signal.

Preferably, the warning device includes a display device, a lighting device, a buzzer or a combination of above devices.

Preferably, the home-style and multi-functional testing hearing-aid device further includes a transmitter, which is disposed in the case and is connected with the processor. The transmitter further connects to an external electronic device. When the processor sends the falling prevention warning signal to the warning device, the processor further sends the falling prevention warning signal to the external electronic device through the transmitter.

Another objective of the present disclosure provides a home-style and multi-functional testing hearing-aid method, which utilizes a falling prevention device in a case, and a deep learning method is utilized to judge a movement status based on a plurality of conventional movement trajectories. When the falling prevention device judges that the movement status does not conform to the plurality of conventional movement trajectories, the falling prevention device generates a falling prevention warning signal. At this time, a warning device receives the falling prevention warning signal and executes a warning operation. In addition, the present disclosure also provides functions such as temperature sensing, pulse measurement, blood pressure measurement and so on. Accordingly, the present disclosure provides a utilizing method of the multi-functional hearing-aid device, which allows the users to use their required functions and to increase the convenience to the users.

To achieve the foregoing objective, the present disclosure provides a home-style and multi-functional testing hearing-aid method by utilizing the above home-style and multi-functional testing hearing-aid device.

The above objects, features, and advantages of the present disclosure may become apparent and be more clearly understood by reference to the detailed description of the embodiments and to the several drawings hereinafter.

### BRIEF DESCRIPTION OF THE DRAWINGS

FIG. 1 is a schematic diagram of the component configuration relation in accordance with the embodiment of the present disclosure.

FIG. 2 is a schematic diagram of the configuration of the temperature sensor in accordance with the embodiment of the present disclosure.

FIG. 3 is a schematic diagram of the configuration of the pulse sensor in accordance with the embodiment of the present disclosure.

FIG. 4 is a schematic diagram of the configuration of the blood pressure sensor in accordance with the embodiment of the present disclosure.

FIG. 5 is a flow chart of the steps in accordance with the embodiment of the present disclosure.

### DETAILED DESCRIPTION OF THE PREFERRED EMBODIMENTS

The advantages, features, and technical method of achieving above objectives will be described in detail with reference to the exemplary embodiments and the accompanying drawings, so as to be easily realized. The present disclosure can be implemented in different ways, so it should not be understood that the present invention is limited to the embodiments set forth herein. On the contrary, for those skilled in the art, the provided embodiments will make the present disclosure more clearly and completely convey the scope of the present disclosure. The present disclosure will only be defined by the added claims.

In addition, the word “comprise” and/or “include” will be acknowledged to imply the inclusion of stated features, areas, bodies, steps, elements and/or components but not the exclusion of one or more other existed or added features, areas, bodies, steps, elements and/or components.

The contents of the present disclosure and the effects that can be achieved will be understood more fully from the detailed description given below and from the accompanying drawings of various embodiments.

In accordance with the embodiment(s) of the present disclosure, the components, process steps, and/or data structures described herein may be implemented using various types of operating systems, computing platforms, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein. Where a method comprising a series of process steps is implemented by a computer or a machine and those process steps can be stored as a series of instructions readable by the machine, they may be stored on a tangible medium such as a computer memory device (e.g., ROM (Read Only Memory), PROM (Programmable Read Only Memory), EEPROM (Electrically Erasable Programmable Read Only Memory), FLASH Memory, Jump Drive, and the like), magnetic storage medium (e.g., tape, magnetic disk drive, and the like), optical storage medium (e.g., CD-ROM, DVD-ROM, paper card and paper tape, and the like) and other known types of program memory.

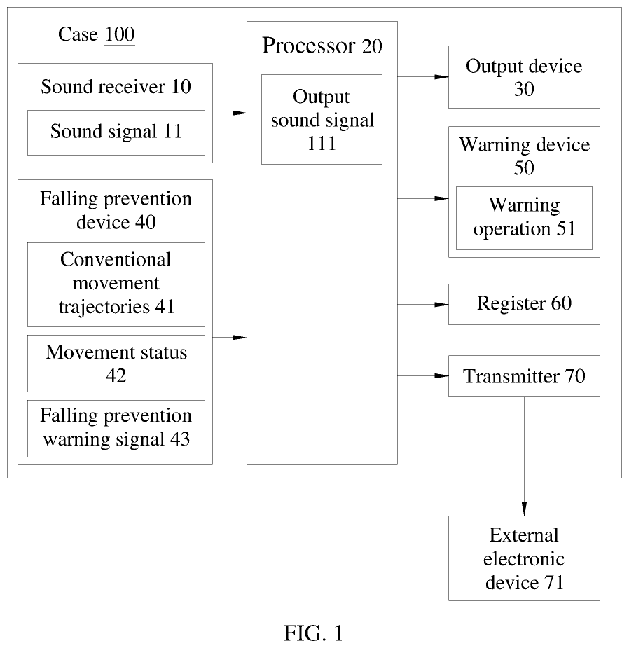

Please refer to FIG. 1, which is a schematic diagram of the component configuration relation in accordance with the embodiment of the present disclosure. As shown in the figure, the present disclosure provides a case 100 with a sound receiver 10, a processor 20, an output device 30, a falling prevention device 40 and a warning device 50 disposed in the case 100. The sound receiver 10 may be a microphone or other related devices that can receive the external sound. The processor 20 may be a central processing unit or other data processing devices. When the processor 20 is connected to the sound receiver 10, a sound signal 11 received by the sound receiver 10 can be received. At this time, the processor 20 amplifies or amplifies and frequency shifts (Utilize a frequency shift method to shift the audio signal 11 to a sound frequency suitable for the user to hear) the sound signal 11 to generate an output sound signal 111. Therefore, the user may hear the output sound signal 111 and will not be unable to hear or cause discomfort to the user due to the inappropriate sound frequencies.

The output device 30 may be connected with the processor 20 to receive the output sound signal 111 and output the output sound signal 111 to the user for listening.

The falling prevention device 40 is disposed in the case 100 and is connected with the processor 20. When the falling prevention device 40 is disposed inside the case 100, since the user walks or moves while hanging the case 100 (That is, the hearing-aid device of the present disclosure), the case 100 usually present as a swing curve or other regular or irregular lines. Thus, in order to collect these lines and to calculate or judge by the collected lines, the falling prevention device 40 may records movements of the case 100 and a deep learning method is utilized to generate a plurality of conventional movement trajectories 41. For example, the lines collected above may be calculated and judged to generate the plurality of conventional movement trajectories 41. Such conventional movement trajectories 41 may be the movement trajectory when the user is walking, the movement trajectory when the user is running, or the movement trajectory when the user is working and so on.

Assuming that the user is walking, the falling prevention device 40 may detect a movement status 42 of the user while hanging the case 100 and judge the movement status 42 of the case 100 based on the plurality of conventional movement trajectories 41. When the falling prevention device 40 judges that the movement status 42 does not conform to the plurality of conventional movement trajectories 41, the falling prevention device 40 generates a falling prevention warning signal 43 in real time and sends the falling prevention warning signal 43 to the processor 20. However, if the falling prevention device 40 judges that the movement status 42 conforms to any one of the plurality of conventional movement trajectories 41, the falling prevention device 40 continues to judge the other movement status 42.

The warning device 50 is disposed in the case 100 and is connected with the processor 20 to receive the falling prevention warning signal 43. When the warning device 50 receives the falling prevention warning signal 43, the warning device 50 executes a warning operation 51. The warning device 50 includes one of a display device, a lighting device, a buzzer or a combination of above devices. Thus, when the warning device 50 is a speaker, it can be presented by a warning sound or a voice. When the warning device 50 is a display screen, a warning image or a graphic can be displayed on the display screen. When the warning device 50 is a lighting device, a flash light can be output while the falling prevention warning signal 43 is received. Obviously, the methods of outputting the falling prevention warning signal 43 by the above components (the display module, the lighting device and the speaker) are not conflict. When the warning device 50 is the combination of the speaker and the display screen, the functions of outputting the warning sound (or the voice) and displaying the warning images or the graphics may be conducted simultaneously.

The home-style and multi-functional testing hearing-aid device of the present disclosure further includes a register 60 and a transmitter 70. The register 60 is disposed in the case 100 and is connected with the processor 20 to store the sound signal 11, the output sound signal 111, the plurality of conventional movement trajectories 41, the movement status 42, the falling prevention warning signal 43 or a combination of above signals. The transmitter 70 is disposed in the case 100 and is connected with the processor 20. The transmitter 70 further connects to an external electronic device 71. When the processor 20 sends the falling prevention warning signal 43 to the warning device 50, the processor 20 further sends the falling prevention warning signal 43 to the external electronic device 71 through the transmitter 70.

Since the conversion or the processing between the digital signals and the analog signals is a conventional technology, in the above signal receiving action or outputting action, the actions known in the prior art will not be repeated.

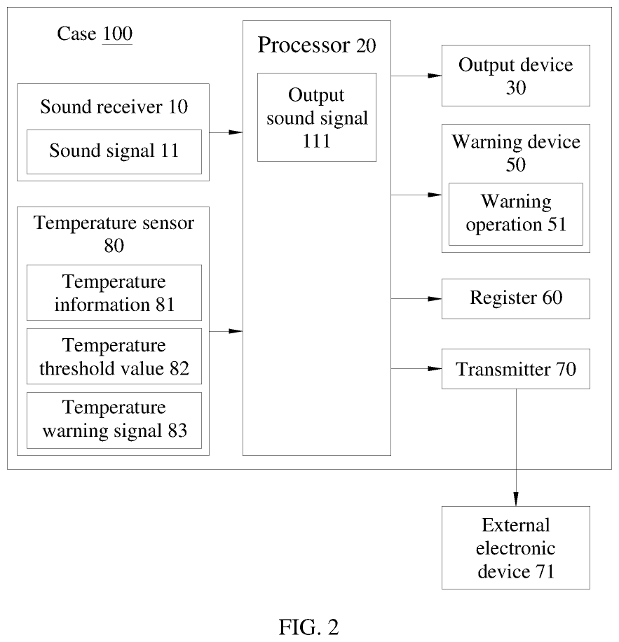

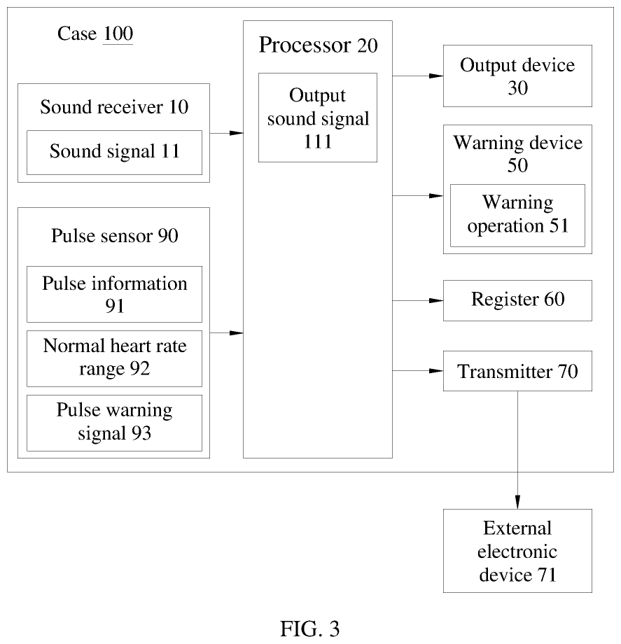

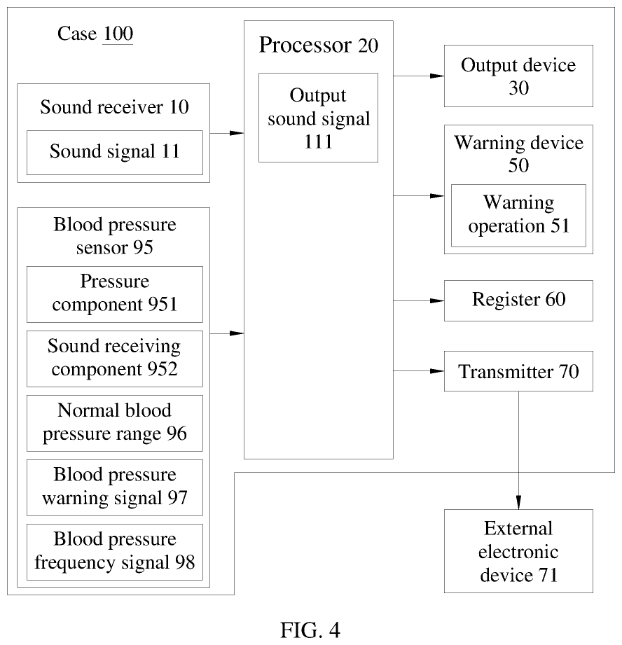

Please refer FIG. 2 to FIG. 4, which are the schematic diagrams of the configuration of the temperature sensor, the pulse sensor and the blood pressure sensor in accordance with the embodiments of the present disclosure. As shown in the figures, in order to monitor the health status of the user, the present disclosure may further include a temperature sensor, a pulse sensor and a blood pressure sensor.

The temperature sensor 80 is disposed on outer surface of the case 100 and is connected with the processor 20. The temperature sensor 80 is in contact or adjacent to the ear to obtain temperature information 81. For example when the temperature sensor 80 is directly contacted with the ear, the temperature information 81 can be obtained by the temperature sensor 80 through the contact portion. When the temperature sensor 80 is adjacent to the ear, the temperature sensor 80 may utilize the sensing wave method to sense the temperature of the ear, so as to obtain the temperature information 81. At this time, the temperature sensor 80 may judges the temperature information 81 based on a temperature threshold value 82 (For example the normal temperature range of the human body is 35.0° C. to 37.0° C.). When a temperature value of the temperature information 81 exceeds the temperature threshold value 82, the temperature sensor 80 generates a temperature warning signal 83 and the temperature warning signal 83 is sent to the processor 20. When the temperature value of the temperature information 81 does not exceed the temperature threshold value 82, the temperature sensor 80 continues to judge the other temperature information 81.

The pulse sensor 90 is disposed on outer surface of the case 100 and is connected with the processor 20. The pulse sensor 90 is in contact to the ear to obtain pulse information 91. For example the pulse sensor 90 may obtain the pulse information 91 by utilizing the physical contact, optical contact or electrical contact methods. At this time, the pulse sensor 90 may judge the pulse information 91 based on a normal heart rate range 92 (For example the pulse beats per minute are about 100-120 times for babies, about 80-100 times for children, and about 60-90 times for adults). When a pulse value of the pulse information 91 exceeds the normal heart rate range 92, the pulse sensor 90 generates a pulse warning signal 93 and the pulse warning signal 93 is sent to the processor 20. When the pulse value of the pulse information 91 does not exceed the normal heart rate range 92, the pulse sensor 90 continues to judge the other pulse information 91.

The blood pressure sensor 95 is disposed on outer surface of the case 100 and is connected with the processor 20. When the blood pressure sensor 95 includes a pressure component 951, the blood pressure sensor 95 may press any portion of the ear by the pressure component 951 and obtain a blood pressure value based on the pressed portion. The blood pressure sensor 95 judges the blood pressure value based on a normal blood pressure range 96 (For example the normal blood pressure range of the human body is less than 120 mmHg for systolic blood pressure and less than 80 mmHg for diastolic blood pressure). When the blood pressure value exceeds the normal blood pressure range 96, the blood pressure sensor 95 generates a blood pressure warning signal 97 and the blood pressure warning signal 97 is sent to the processor 20. When the blood pressure value does not exceed the normal blood pressure range 96, the blood pressure sensor 95 continues to judge the other blood pressure value.

When the blood pressure sensor 95 includes a sound receiving component 952, the pressure component 952 may contact any portion of the ear and the blood pressure sensor 95 converts the blood pressure frequency signal 98 to the blood pressure value. The blood pressure sensor 95 judges the blood pressure value based on a normal blood pressure range 96. When the blood pressure value exceeds the normal blood pressure range 96, the blood pressure sensor 95 generates a blood pressure warning signal 97 and the blood pressure warning signal 97 is sent to the processor 20. When the blood pressure value does not exceed the normal blood pressure range 96, the blood pressure sensor 95 continues to judge the other blood pressure value.

When the warning device 50 receives the temperature warning signal 83, the pulse warning signal 93 or the blood pressure warning signal 97, the warning device 50 executes the warning operation 51 as described above.

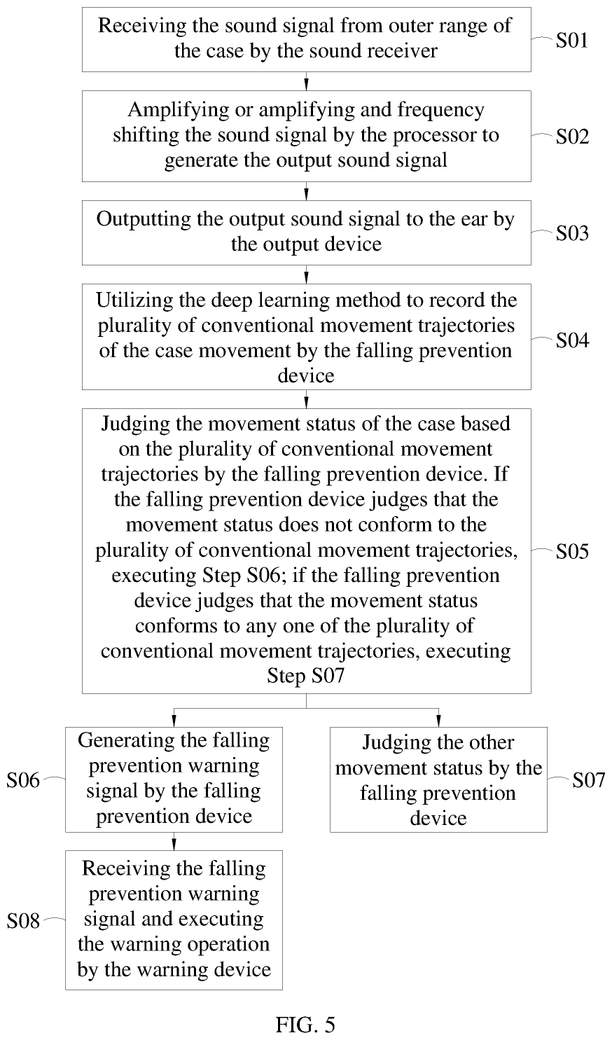

Please refer to FIG. 5, which is a flow chart of the steps in accordance with the embodiment of the present disclosure. As shown in the figure, the present disclosure may achieve the above functions of the home-style and multi-functional testing hearing-aid device by the following process steps (S01-S04):

Step S01: Receiving the sound signal from outer range of the case by the sound receiver.

Step S02: Amplifying or amplifying and frequency shifting the sound signal by the processor to generate the output sound signal.

Step S03: Outputting the output sound signal to the ear by the output device.

Step S04: Utilizing the deep learning method to record the plurality of conventional movement trajectories of the case movement by the falling prevention device.

Step S05: Judging the movement status of the case based on the plurality of conventional movement trajectories by the falling prevention device. If the falling prevention device judges that the movement status does not conform to the plurality of conventional movement trajectories, executing Step S06; if the falling prevention device judges that the movement status conforms to any one of the plurality of conventional movement trajectories, executing Step S07.

Step S06: Generating the falling prevention warning signal by the falling prevention device.

Step S07: Judging the other movement status by the falling prevention device.

Step S08: Receiving the falling prevention warning signal and executing the warning operation by the warning device.

It is noted that the above steps S01-S03 are the hearing-aid processes to receive the outer sound and to execute the amplify operation; steps S04-S08 are the detecting and warning processes to prevent falling. Thus, there is no specific sequence between the steps S1-S03 and the steps S04-S08. That is, the process may execute the steps S01-S03 first and then the steps S04-S08. On the contrary, the process may execute the steps S04-S08 first and then the steps S01-S03.

The present disclosure provides a hearing-aid device with multiple functions, so that the user can use it based on their required function and the convenience to the user can be increased.

While the means of specific embodiments in present disclosure has been described by reference drawings, numerous modifications and variations could be made thereto by those skilled in the art without departing from the scope set forth in the claims.

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

In this application, apparatus elements described as having particular attributes or performing particular operations are specifically configured to have those particular attributes and perform those particular operations. Specifically, a description of an element to perform an action means that the element is configured to perform the action. The configuration of an element may include programming of the element, such as by encoding instructions on a non-transitory, tangible computer-readable medium associated with the element.

What is claimed is:

1. A home-style and multi-functional testing hearing-aid device, hanging on an ear and comprising:
a case;
a sound receiver disposed in the case and receiving a sound signal from outer range of the case;
a processor disposed in the case and connected with the sound receiver to receive the sound signal, and the sound signal being amplified or amplified and frequency shifted to generate an output sound signal;
an output device disposed in the case and connected with the processor to receive and output the output sound signal to the ear;
a falling prevention device disposed in the case and connected with the processor, the falling prevention device recording movements of the case, and a deep learning method being utilized to generate a plurality of conventional movement trajectories based on lines collected by recording the movements of the case and to judge a movement status of the case based on the plurality of conventional movement trajectories, wherein when the movement status does not conform to the plurality of conventional movement trajectories, the falling prevention device generates a falling prevention warning signal and sends the falling prevention warning signal to the processor, wherein when the falling prevention device judges that the movement status conforms to any one of the plurality of conventional movement trajectories, the falling prevention device continues to judge other movement status; and
a warning device disposed in the case and connected with the processor to receive the falling prevention warning signal, the warning device executing a warning operation when the warning device receives the falling prevention warning signal.

2. The home-style and multi-functional testing hearing-aid device of claim 1, further comprising:
a temperature sensor disposed on outer surface of the case and connected with the processor, the temperature sensor being in contact or adjacent to the ear to obtain temperature information and the temperature sensor judging the temperature information based on a temperature threshold value, wherein when a temperature value of the temperature information exceeds the temperature threshold value, the temperature sensor generates a temperature warning signal and the temperature warning signal is sent to the processor, wherein when the temperature value of the temperature information does not exceed the temperature threshold value, the temperature sensor continues to judge other temperature information;
wherein the warning device executes the warning operation when the warning device receives the temperature warning signal.

3. The home-style and multi-functional testing hearing-aid device of claim 1, further comprising:
a pulse sensor disposed on outer surface of the case and connected with the processor, the pulse sensor being in contact to the ear to obtain pulse information and the pulse sensor judging the pulse information based on a normal heart rate range, wherein when a pulse value of the pulse information exceeds the normal heart rate range, the pulse sensor generates a pulse warning signal and the pulse warning signal is sent to the processor, wherein when the pulse value of the pulse information does not exceed the normal heart rate range, the pulse sensor continues to judge other pulse information;
wherein the warning device executes the warning operation when the warning device receives the pulse warning signal.

4. The home-style and multi-functional testing hearing-aid device of claim 1, further comprising:
a register disposed in the case and connected with the processor to store the sound signal, the output sound signal, the plurality of conventional movement trajectories, the movement status, the falling prevention warning signal or a combination of above signals.

5. The home-style and multi-functional testing hearing-aid device of claim 1, further comprising:
a blood pressure sensor disposed on outer surface of the case and connected with the processor, the blood pressure sensor comprising a pressure component and the pressure component pressing any portion of the ear to obtain a blood pressure value and the blood pressure sensor judging the blood pressure value based on a normal blood pressure range, wherein when the blood pressure value exceeds the normal blood pressure range, the blood pressure sensor generates a blood pressure warning signal and the blood pressure warning signal is sent to the processor, wherein when the blood pressure value does not exceed the normal blood pressure range, the blood pressure sensor continues to judge other blood pressure value.

6. The home-style and multi-functional testing hearing-aid device of claim 5, wherein the warning device executes the warning operation when the warning device receives the blood pressure warning signal.

7. The home-style and multi-functional testing hearing-aid device of claim 1, further comprising:
a blood pressure sensor disposed on outer surface of the case and connected with the processor, the blood pressure sensor comprising a sound receiving component and the sound receiving component being in contact to any portion of the ear to obtain a blood pressure frequency signal, the blood pressure sensor converting the blood pressure frequency signal to a blood pressure value and the blood pressure sensor judging the blood pressure value based on a normal blood pressure range, wherein when a blood pressure value exceeds the normal blood pressure range, the blood pressure sensor generates a blood pressure warning signal and the blood pressure warning signal is sent to the processor, wherein when the blood pressure value does not exceed the normal blood pressure range, the blood pressure sensor continues to judge other blood pressure value.

8. The home-style and multi-functional testing hearing-aid device of claim 7, wherein the warning device executes the warning operation when the warning device receives the blood pressure warning signal.

9. The home-style and multi-functional testing hearing-aid device of claim 1, wherein the warning device comprising a display device, a lighting device, a buzzer or a combination of above devices.

10. The home-style and multi-functional testing hearing-aid device of claim 1, further comprising:
a transmitter disposed in the case and connected with the processor, the transmitter further connecting to an external electronic device, wherein when the processor sends the falling prevention warning signal to the warning device, the processor further sends the falling prevention warning signal to the external electronic device through the transmitter.

11. A home-style and multi-functional testing hearing-aid method by utilizing the home-style and multi-functional testing hearing-aid device of claim 1, the method comprising the following steps:
receiving the sound signal from outer range of the case by the sound receiver;
amplifying or amplifying and frequency shifting the sound signal by the processor to generate the output sound signal;
outputting the output sound signal to the ear by the output device;
utilizing the deep learning method to record the plurality of conventional movement trajectories of the case movement by the falling prevention device;
judging the movement status of the case based on the plurality of conventional movement trajectories by the falling prevention device;
generating the falling prevention warning signal by the falling prevention device; and
receiving the falling prevention warning signal and executing the warning operation by the warning device.
